(19) United States
(12) Patent Application Publication
(10) Pub. No.: US 2024/0420829 A1
(43) Pub. Date: Dec. 19, 2024

(54) BLOOD DONOR OPTIMIZATION SYSTEM

(71) Applicant: BLOOD BANK COMPUTER SYSTEMS, INC., Bonney Lake, WA (US)

(72) Inventor: BRIAN FORBIS, Bonney Lake, WA (US)

(21) Appl. No.: 18/739,689

(22) Filed: Jun. 11, 2024

Related U.S. Application Data
(60) Provisional application No. 63/472,719, filed on Jun. 13, 2023.

(51) Int. Cl. G16H 40/20

(52) U.S. Cl. CPC G16H 40/20

(57) ABSTRACT

Disclosed herein is a blood donor optimization system (BDOS) that enables administrators to define various donation procedures and the corresponding donor qualifications. Furthermore, blood centers can dynamically adjust their collection goals in real-time using the BDOS. When a donor visits any blood center or participates in a blood drive within the region, the BDOS instantly evaluates the donor's qualifications for each available procedure. Based on the blood center's defined goals, the system identifies and presents the ideal procedure for the donor during that visit. Additionally, the BDOS provides a ranking of alternative procedures, indicating the most impactful options to the least helpful. As a result, this optimized approach increases the frequency of obtaining the most suitable donation type from each donor, ensuring an efficiently managed blood supply that meets the needs of patients in a timely manner.

### CROSS-REFERENCE TO RELATED APPLICATIONS

This application claims priority to U.S. Provisional Application Ser. No. 63/472,719, filed Jun. 13, 2023, the entire contents of which are hereby incorporated by reference in their entirety.

### FIELD OF THE INVENTION

The present invention discloses a blood donor optimization for enhancing blood collection efficiency and supply management.

### BACKGROUND

Blood donation plays a vital role in the United States healthcare industry. As transfusion medicine research advances, significant discoveries have revolutionized the blood donation process. From the early whole blood vein-to-vein transfusion approach to the modern specialized apheresis donations, the potential for both donors and patients has greatly increased. However, these advancements have introduced complexities in determining the appropriate donation type from the right donor at the right time. Moreover, blood centers have transitioned from hospital-based units to organizations serving multiple hospitals across extensive geographic regions. Consequently, when urgent blood needs arise, the ability to swiftly adapt collection efforts across the entire region becomes crucial.

Currently, determining the optimal blood product to collect from a donor is a time-consuming process. Blood centers often rely on a generalized, one-size-fits-all approach, rather than tailoring donations to maximize their impact. Additionally, these centers face challenges in disseminating procedural changes swiftly enough to effectively respond to emergency cases throughout their region. Therefore, a need currently exists for quickly identifying the optimal blood product to collect from donors.

### SUMMARY

In order to quickly identifying the optimal blood product to collect from donors, the present invention discloses a blood donor optimization system (BDOS) that enables administrators to define various donation procedures and the corresponding donor qualifications. Furthermore, blood centers can dynamically adjust their collection goals in real-time using the BDOS. When a donor visits any blood center or participates in a blood drive within the region, the BDOS instantly evaluates the donor's qualifications for each available procedure. Based on the blood center's defined goals, the system identifies and presents the ideal procedure for the donor during that visit. Additionally, the BDOS provides a ranking of alternative procedures, indicating the most impactful options to the least helpful. As a result, this optimized approach increases the frequency of obtaining the most suitable donation type from each donor, ensuring an efficiently managed blood supply that meets the needs of patients in a timely manner.

### BRIEF DESCRIPTION OF THE DRAWINGS

FIG. 1 depicts a screenshot of a goals administration page for entering a new blood donation goal.

FIG. 2 depicts an example blood donor profile page.

FIG. 3 depicts an example screen showing tallies for all goals.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

The present invention discloses a blood donor optimization system (BDOS) that enables administrators to define various donation procedures and the corresponding donor qualifications. Furthermore, blood centers can dynamically adjust their collection goals in real-time using the BDOS. When a donor visits any blood center or participates in a blood drive within the region, the BDOS instantly evaluates the donor's qualifications for each available procedure. Based on the blood center's defined goals, the system identifies and presents the ideal procedure for the donor during that visit. Additionally, the BDOS provides a ranking of alternative procedures, indicating the most impactful options to the least helpful. As a result, this optimized approach increases the frequency of obtaining the most suitable donation type from each donor, ensuring an efficiently managed blood supply that meets the needs of patients in a timely manner.

By implementing the BDOS, blood centers can enhance their efficiency, responsiveness, and the overall quality of blood supply, ultimately benefiting patients across the region in need of life-saving transfusions.

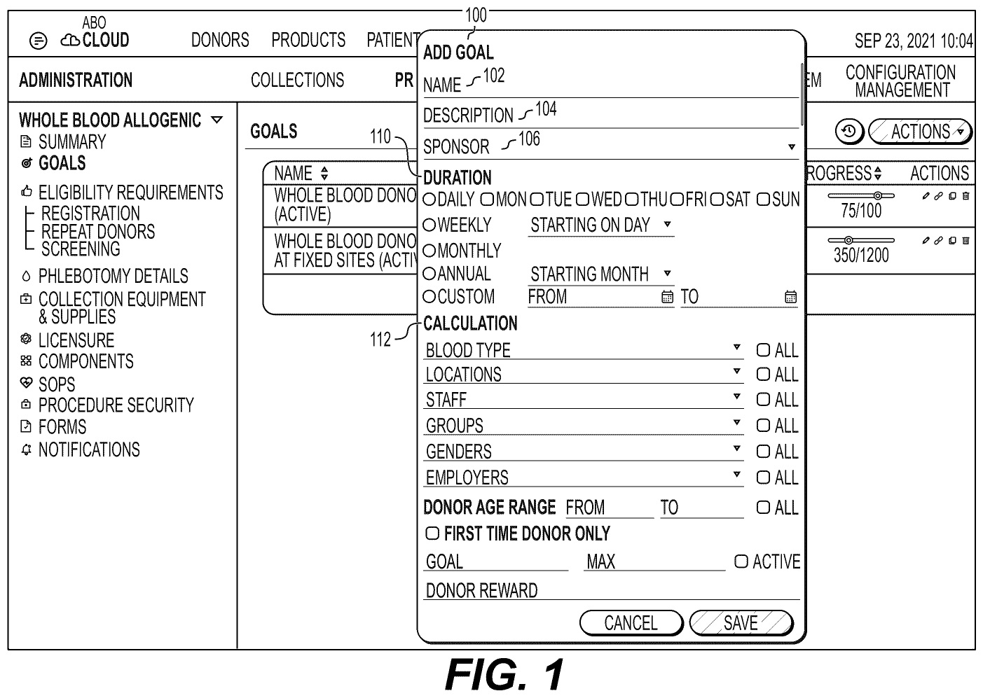

FIG. 1 depicts a screenshot of a goals administration page for entering a new blood donation goal. As depicted, each goal 100 includes a name 102, description 104, and sponsor 106 (e.g., a particular employee).

The goal 100 may include a duration 110 for collection. For example, the goal 100 may repeat daily or on specific days. Further, the duration 110 may be set to occur, weekly, monthly, annually, etc. or for a specified date range.

Each goal 100 may set a variety of requirements 112 used in tracking the goal 100. Fields for the requirements 112 may include, but are not limited to, blood type, location, staff, groups, genders, employers, donor age range, first time donor status, or donor reward. Each goal is further defined with filters that determine if a successfully completed procedure counts toward the goal. These filters may include specific blood center locations or staff and may also include characteristics of the donor: gender, age range, blood type, group membership, employer, or first-time donor status. Once all of the requirements 112 have been entered, the goal 100 can be saved. All of the various goals 100 for blood collection can be entered and monitored via the BDOS. Then, when donors arrive for a blood donation, the various goals that have been entered can be checked by the BDOS to determine the best fit for the donor.

The procedure goals engine of the BDOS indicates the most ideal procedures for an allogeneic donor at the time of donor registration. The ideal procedures are calculated and ranked by a complex algorithm that uses customizable goal criteria defined by the blood center. The advantage the Procedure Goals Engine brings to a blood center is to ensure the center's collection goals and business needs are actively managed during the donation workflow.

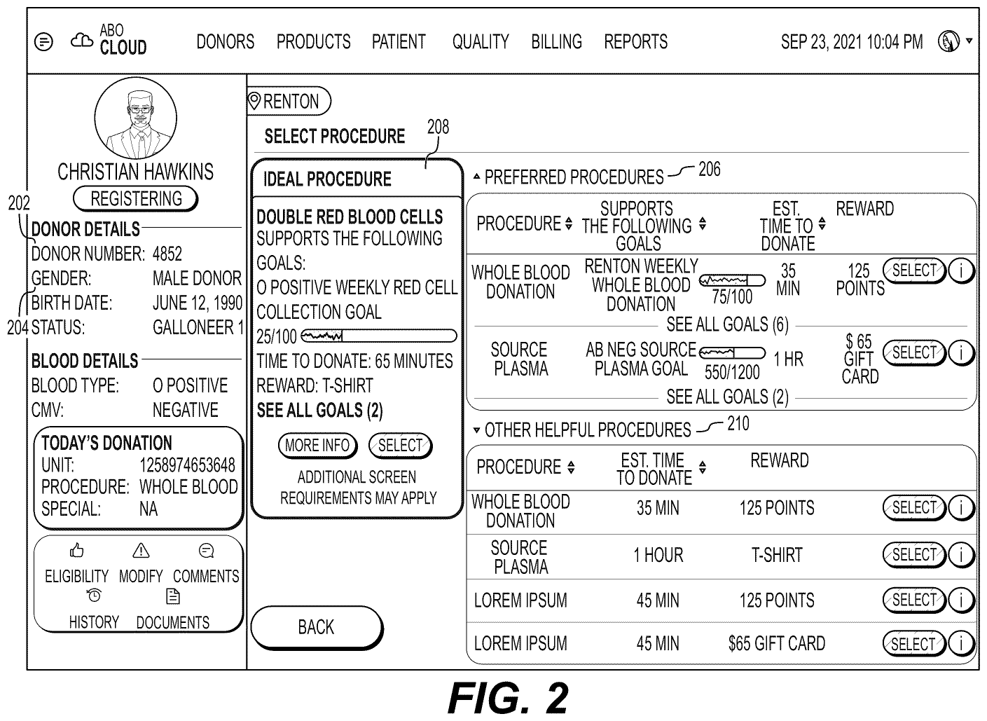

When a donor arrives for donation, the donor profile is pulled up or created in the BDOS. An example screenshot of a donor profile page is depicted in FIG. 2. Each donor is assigned a donor identification 202 which is unique to each particular donor. Other identifying information 204 is also collected which may include gender, birthdate, donor status, blood type, CMV status, etc.

Once the donor is registered, an ideal procedure 208 is identified for the donor based on current needs and the donor information. For example, FIG. 2. depicts that the ideal procedure 208 for the depicted donor is for double red blood cells. Information about the ideal procedure 208 is depicted and can be used to inform the donor about information regarding the ideal procedure 208 such as time for donation, possible rewards, etc. The administrator can select the ideal procedure 208 for the donor, or the administrator can select a preferred procedure 206, or other helpful procedure 208. The other helpful procedures 210 are procedures that the donor qualifies for, but are not a good fit as the ideal procedure 208 (which is the procedure corresponding to the top ranked goal for the donor).

The ideal procedure 208, preferred procedures 206, and the other helpful procedures 210 are identified by analyzing each goal in the goal list and comparing it to the donor information. For example, the donor information 204 is compared to each of the goals. Goal requirements are checked for each donor including location, blood type, donor group, gender, and age. If the donor is a match, it is next checked if the goal's 100 max count has been reached (e.g., number of required donations). If the goal has not been met and the procedure associated with the goal is selected for the donor, the tick count for the goal is increased by one and the goal is eventually closed when the tick count reaches the max count. Other goal information may also be checked such as the requirement of first time or recurring donors, specific screening requirements (e.g., for different diseases) have been met.

After the donor information has been compared to all of the goals, a list of matching goals 100 is determined and ranked. The top scoring goal 100 is assigned as the ideal procedure 208 and the remaining matching goals are listed in other helpful procedures 210. The identification of the ideal procedure 208 for each donor helps to ensure that all the goals 100 are being fulfilled in the most efficient manner by the BDOS.

In a preferred embodiment, the goal with the fewest days remaining will be given top priority as the listed ideal procedure 208. If two-or-more goals are tied for the fewest days remaining, the one with the highest number of units remaining until the goal is reached will be assigned a higher score. The procedures 210 with goals for which the donor is eligible will be displayed in the ‘Preferred Procedures’ section in order of highest goal score to lowest. Any other procedures performed at this location for which the donor is eligible will be displayed as other helpful procedures 210.

Once registered for a procedure, if it is later determined that the donor does not meet the screening requirements for the selected procedure and before canceling the donation process, the BDOS performs a second evaluation of goals and if the donor is still eligible for other procedures, the algorithm is performed again and a new set of Ideal procedures 208, Preferred procedures 206, and Other Helpful Procedures 210 is presented to the administrator.

Once the donor successfully completes the procedure, it counts toward the specific goal only if all the filter criteria is met. Based on the blood center's customized goals, there may be more than one goal for the procedure in which the successful completion of the procedure may count towards. If the donor does not meet the criteria for a procedure goal, the procedure is still displayed for the user to select but is not highlighted as an Ideal or Preferred Procedure. If selected, the donor's donation does not count towards filling the goal.

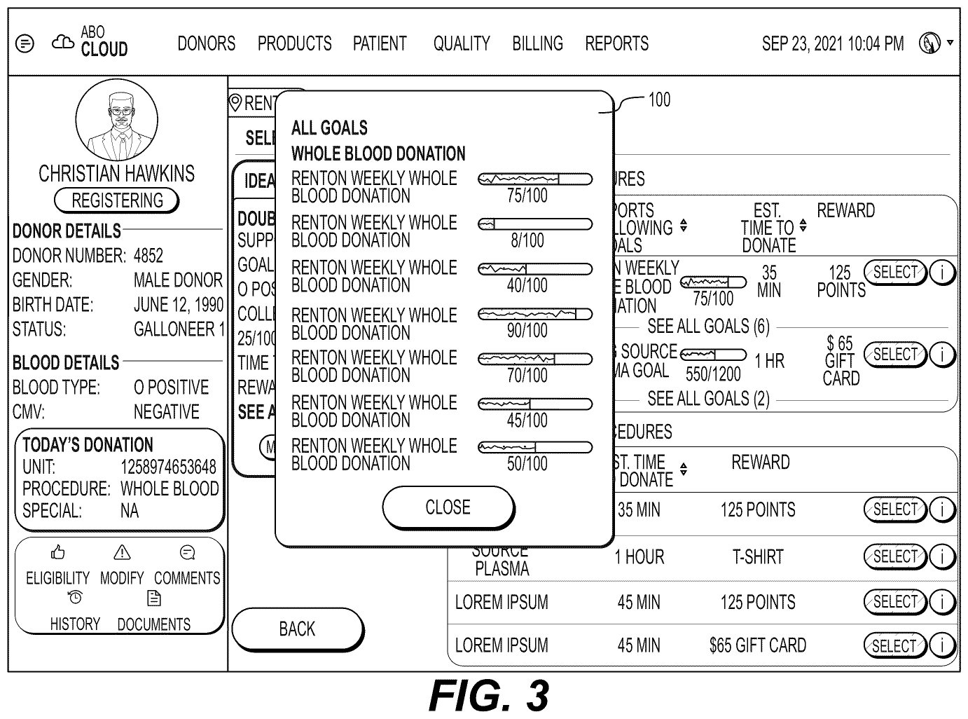

If goals are defined, but there are no goals applicable to the donor and location combination, all allogeneic procedures supported at this location for which the donor is eligible are displayed as other helpful procedures 210. If goals are not defined by the blood center, a single list of ‘Available Procedures’ will be displayed. This list will include every allogeneic procedure supported at the location for which the donor is eligible. Additional information about global tallies for each goal all goals 100 can also be viewed using BDOS as depicted in FIG. 3.

While specific embodiments of the invention have been described above, it will be appreciated that the invention may be practiced other than as described. The embodiment(s) described, and references in the specification to “one embodiment,” “an embodiment,” “an example embodiment,” “some embodiments,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A method for operating a blood donor optimization system (BDOS) comprising:
defining a plurality of collection procedures,
wherein each collection procedure comprises a goal and donor requirements;
receiving, from a donor, donor qualifications;
comparing the donor qualifications to the donor requirements of the plurality of collection procedures;
producing a ranked list of the plurality of collection procedures based on the comparing;
presenting a first collection procedure from the ranked list to the donor;
collecting a blood donation from the donor in response to the user accepting the first collection procedure; and
updating the goal of the first collection procedure in response to the collecting.

2. The method according to claim 1, wherein an administrator of the BDOS can adjust the goal or the donor requirements of each collection procedure of the plurality of collection procedures in real time.

3. The method according to claim 1, wherein the goal includes a time duration, and
wherein the time duration is set to repeat are regular intervals.

4. The method according to claim 1, wherein the donor requirements include a blood type, a location, a staffing level, a gender, an employer, an age range, a first time donor status, or a donor reward.

5. The method according to claim 1, further comprising:
excluding a subset of collection procedures from the plurality of collection procedures from the comparison if a goal percentage is above a predefined threshold.

6. The method according to claim 1, further comprising:
providing a reward to the donor if the collecting is successful.

7. The method according to claim 1, further comprising:
presenting a list of alternative collection procedures from the ranked list to the donor to the donor.

8. The method according to claim 1, wherein the ranked list includes a score assigned to each collection procedure of the plurality of collection procedures.

9. The method according to claim 8, wherein the score of the first collection procedure is also presented to the user along with the first collection procedure.

10. The method according to claim 1, wherein the ranked list is divided into ideal procedures and preferred procedures.

11. A method for operating a blood donor optimization system (BDOS) comprising:
defining a plurality of collection procedures,
wherein each collection procedure comprises a goal and donor requirements;
receiving, from a donor, donor qualifications;
comparing the donor qualifications to the donor requirements of the plurality of collection procedures;
producing a ranked list of the plurality of collection procedures based on the comparing;
presenting a first collection procedure from the ranked list and a list of secondary collection procedures from the ranked list to the donor,
wherein the list of secondary collection procedures each has a lower completion score than the first collection procedure;
collecting a blood donation from the donor in response to a user selection of the first collection procedure or from the list of secondary collection procedure; and
updating the goal of collection procedure corresponding to the user selection in response to the collecting.
